(19) United States
(12) Patent Application Publication
(10) Pub. No.: US 2014/0243611 A1
(43) Pub. Date: Aug. 28, 2014

(54) REHABILITATION DEVICE

(71) Applicants: Akihiro Ishikawa, Kyoto (JP); Yoshihiro Inoue, Kyoto (JP)

(72) Inventors: Akihiro Ishikawa, Kyoto (JP); Yoshihiro Inoue, Kyoto (JP)

(21) Appl. No.: 14/131,413

(22) PCT Filed: Aug. 10, 2011
(86) PCT No.: PCT/JP2011/068301

§ 371 Date: Jan. 7, 2014

(51) Int. Cl. A61B 5/11; A61B 5/00; A61B 5/16

(52) U.S. Cl. CPC A61B 5/11; A61B 5/16; A61B 5/0059
USPC 600/301

(57) ABSTRACT

A rehabilitation device includes a head mounted unit disposed on the scalp surface of a subject, a brain measurement data acquisition unit which acquires brain measurement data relating to a brain activity, a determination unit which determines whether the subject's brain can recall the brain activity corresponding to an exercise which is a subject for rehabilitation based on the brain measurement data, a motion sensor attached to a part of the subject's body, a body measurement data acquisition unit which acquires body measurement data relating to a body movement of a part of the subject's body from the motion sensor, and a warning unit which gives a warning to stop the body movement of the part of the subject's body when there is a body movement that does not correspond to the exercise which is the subject for rehabilitation based on the body measurement data.

### TECHNICAL FIELD

The present invention relates to a rehabilitation device and more specifically relates to a rehabilitation device for rehabilitation of patients (subjects) in which partial paralysis of the body, such as that caused by the after effects of cerebral stroke, has occurred.

### BACKGROUND

In recent years, brain function imaging devices that conveniently make noninvasive measurements using light have been developed for observing the state of activity in the brain. With these optical brain function imaging devices, near infrared light of three different wavelengths, λ₁, λ₂ and λ₃ (for example, 780 nm, 805 nm and 830 nm) irradiates the brain by means of a light transmitting probe disposed on the surface of the scalp of a subject, and further, the intensity (information on amount of light received) of the near infrared light of the various wavelengths λ₁, λ₂, and λ₃ released by the brain, A(λ₁), A(λ₂) and A(λ₃), is detected for each by a light receiving probe disposed on the surface of the scalp.

Furthermore, to find the product [oxyHb] of the oxyhemoglobin concentration and the optical path length and the product [deoxyHb] of the deoxyhemoglobin concentration and the optical path length from the information on the amount of light received A(λ₁), A(λ₂) and A(λ₃), the simultaneous equations shown in relational equations (1), (2) and (3), for example, are created using the modified Beer-Lambert law, and these simultaneous equations are solved (for example, see non-patent reference 1). Furthermore, the product ([oxyHb]+[deoxyHb]) of the total hemoglobin concentration and the optical path length is calculated from the product [oxyHb] of the oxyhemoglobin concentration and the optical path length and the product [deoxyHb] of the deoxyhemoglobin concentration in the optical path length.

A(λ₁)=EO(λ₁)×[oxyHb]+Ed(λ₁)×[deoxyHb] (1)

A(λ₂)=EO(λ₂)×[oxyHb]+Ed(λ₂)×[deoxyHb] (2)

A(λ₃)=EO(λ₃)×[oxyHb]+Ed(λ₃)×[deoxyHb] (3)

Moreover, EO(λₘ) is the light absorbance coefficient for oxyhemoglobin in light of a wavelength λₘ and Ed(λₘ) is the light absorbance coefficient for deoxyhemoglobin in light of a wavelength

Here, the relationship between the distance (channel) between the light transmitting probe and light receiving probe and the region being measured will be described. FIG. 4(a) is a cross-sectional view showing the relationship between a pair of a light transmitting probe and a light receiving probe and the region being measured, and FIG. 4(b) is a plan view for FIG. 4(a).

A light transmitting probe 12 is pushed against a light transmission point T on the surface of the scalp of the subject, and a light receiving probe 13 is also pressed against a light receiving point R on the surface of the scalp of the subject. Furthermore, light is irradiated from the light transmitting probe 12 and also light that is released from the surface of the scalp is incident to the light receiving probe 13. At this time the light that reaches the light receiving point R on the surface of the scalp is light that has passed through a banana shape (measurement region) out of the light irradiated from the transmission point T on the surface of the scalp. Thus, even in the measurement region, information on the amount of light received A(λ₁), A(λ₂) and A(λ₃) is obtained for a site S being measured in the subject, which is at a depth L/2, which is half the distance along a line that joins the transmission point T and the light receiving point R by the shortest distance along the surface of the scalp, from a middle point M on the line L, which joins the light transmission point T and the light receiving point R by the shortest distance along the surface of the scalp of the subject.

In addition, in order to measure the product [oxyHb] of the oxyhemoglobin concentration and the optical path length, the product [deoxyHb] of the deoxyhemoglobin concentration and the optical path length and the product ([oxyHb]+[deoxyHb]) of the total hemoglobin concentration and the optical path length for measurement sites in a plurality of locations in the brain with a brain function imaging device, a near-infrared spectrometer or the like, for example, is used (for example, see patent reference 1).

FIG. 5 is a block diagram showing an example of a schematic constitution for a conventional near-infrared spectrometer. Moreover, several light transmitting optical fibers, several light receiving optical fibers and the like are omitted to facilitate visualization. The near-infrared spectrometer 101 has a housing 11 with a cuboid shape.

Inside the housing 11, a light source 2 that irradiates light, a light source drive mechanism 4 that drives the light source 2, a light detector 3 that detects light, an A/D (A/D converter) 5, a control unit 21 for transmitting and receiving light, a control unit 122 for analysis and memory 23 are provided. In addition, outside the housing 11, 64 light transmitting probes 12, 64 light receiving probes 13, 64 light transmitting optical fibers 14, 64 light receiving optical fibers 15, a display device 26 that has a monitor screen 26a and the like, and a keyboard (input device) 27 are provided.

The light source drive mechanism 4 drives the light source 2 by a drive signal input from the control unit 21 for transmitting and receiving light. The light source 2 includes semiconductor lasers LD1, LD2, LD3 and the like that can output near infrared light in the three different wavelengths λ₁, λ₂, and λ₃.

The light detector 3 is a detector that outputs light receiving signals (information on the amount of light received) A(λ₁), A(λ₂) and A(λ₃) to the control unit 21 for transmitting and receiving light via the A/D 5 by detecting each of near infrared light.

The light transmitting optical fibers 14 and light receiving optical fibers 15 are tubular shaped with a diameter of 2 mm and a length of 2 m to 10 m and can propagate near infrared light in the axial direction. Near infrared light incident from one end part passes through the inside thereof and exits from the other end part. Near infrared light incident from the other end part passes through the inside thereof and exits from the one end part.

The two end parts of a single light transmitting optical fiber 14 are connected to one light transmitting probe 12 and one semiconductor laser LD1, LD2, LD3 for the light source 2 such that there is separation by a set length (2 m-10 m).

The two end parts of a single light receiving optical fiber 15 are connected to one light receiving probe 13 and one photomultiplier tube for the light detector 3 such that there is separation by a set length (2 m-10 m).

A holder (light sending and receiving unit) 30 is used in this near-infrared spectrometer 101 to make contact between the 64 light transmitting probes 12 and the 64 light receiving probes 13 and the surface of the scalp of the subject in a prescribed arrangement. FIG. 2 is a plan view showing an example of the holder 30 into which the 64 light transmitting probes and 64 light receiving probes are inserted.

The light transmitting probes 12T1-12T64 and the light receiving probes 13R1-13R64 are arranged so as to alternate; 16 in the vertical direction and 16 in the horizontal direction. Thus, the spacing between the light transmitting probes 12 and the light receiving probes 13 is fixed, and information on the amount of light received A(λ₁), A(λ₂) and A(λ₃), which is at a specific depth from the surface of the scalp, is obtained. Moreover, a channel set at 30 mm is typically used, and obtaining information on the amount of light received A(λ₁), A(λ₂) and A(λ₃) for a depth of 15 mm to 20 mm from the middle point of the channel can be considered when the channel is 30 mm In other words, a position at a depth of 15 mm to 20 mm from the surface of the scalp substantially corresponds to a site on the brain surface, and information on the amount of light received A(λ₁), A(λ₂) and A(λ₃) relating to brain activity is obtained.

The control unit 21 for transmitting and receiving light outputs the drive signal for transmitting light to one light transmitting probe 12 to the light source drive mechanism 4 at a prescribed time based on a control table stored in the memory 23, and a light receiving signal (information on the amount of light received) for light received by a light receiving probe 13 is detected by the light detector 3.

As a result, with a plan view as shown in FIG. 2, collection of a total of 232 sets (S1-S232) of information on the amount of light received A(λ₁), A(λ₂) and A(λ₃) is carried out. Furthermore, the control unit 122 for analysis finds the product [oxyHb] of the oxyhemoglobin concentration and the optical path length, the product [oxyHb] of the oxyhemoglobin concentration and optical path length and the product ([oxyHb]+[deoxyHb]) of the total hemoglobin concentration and the optical path length from the intensity of the light of the various wavelengths (oxyhemoglobin absorption wavelength and deoxyhemoglobin absorption wavelength) passing through using relational equations (1), (2) and (3) based on the total of 232 sets of information on the amount of light received A(λ₁), A(λ₂) and A(λ₃).

In recent years, progress has been made on research concerning methods and devices for providing effective recovery of paralyzed parts (parts of the body) in patients that have paralysis caused strokes and the like. Thus, rehabilitation devices equipped with a near-infrared spectrometer 101 that can obtain the product [oxyHb] (brain measurement data) of the oxyhemoglobin concentration and the optical path length in patients have been proposed (for example, see patent reference 3).

Thus, when rehabilitation is carried out for the patient, the rehabilitation device can execute graphical display of the fact that the brain of the patient is recalling brain activity and the extent to which the brain of the patient is recalling the brain activity by determining whether or not the brain of the patient is recalling brain activity corresponding to the exercises targeted by the rehabilitation based on the product [oxyHb] of oxyhemoglobin concentration and the optical path length.

### DOCUMENTS OF PRIOR ART

### Patent References

Patent Reference 1: Published Unexamined Patent Application No. 2001-337033

Patent Reference 2: Published Unexamined Patent Application No. 2009-077841

Patent Reference 3: Published U.S. Pat. No. 4,618,795

### Non-Patent References

Non-patent Reference 1: Factors affecting the accuracy of near-infrared spectroscopy concentration calculations for focal changes in oxygenation parameters, Neurolmage 18, 865-879, 2003

### SUMMARY OF THE INVENTION

### Problems to be Solved

However, even if brain activity corresponding to exercises that are the target of the rehabilitation is recalled in the brain of the patient when rehabilitation is carried out, the patient has not been moving the paralyzed part for a long time. Therefore, there have been instances when bodily movement (bodily movement of parts different from the paralyzed part, arm or the like) that does not correspond to the exercises (exercise of the paralyzed part) targeted by the rehabilitation is caused. Therefore, artifacts and noise caused by bodily movement that does not correspond to the exercises targeted by the rehabilitation may enter the brain measurement data, and the determination results of the rehabilitation devices may not be accurate.

Thus, it is an object of the present invention to provide a rehabilitation device that can obtain accurate brain measurement data into which artifacts and noise do not enter and effectively carry out rehabilitation of a subject by giving a warning when bodily movement (bodily movement of parts different from the paralyzed part, arm or the like) that does not correspond to the exercises targeted by the rehabilitation has been caused.

### Means to Solve the Problems

The rehabilitation device of the present invention, which was brought about to solve the problems above, is a rehabilitation device comprising a head mounted unit that is disposed on the surface of the scalp of a subject, a brain measurement data acquisition unit that obtains brain measurement data concerning brain activity by controlling the head mounted unit, and a determination unit that determines whether or not the brain of the subject can recall brain activity corresponding to exercises of a paralyzed part targeted by rehabilitation based on the brain measurement data and further comprises a motion sensor attached to part of the subject's body other than the paralyzed part, a body measurement data acquisition unit that obtains body measurement data concerning bodily movement of the part of the subject's body from the motion sensor and a warning unit that provides a warning to stop bodily movement of the part of the subject's body when, based on the body measurement data, there is detection of bodily movement that does not correspond to the exercises targeted by the rehabilitation.

### Effects of the Invention

According to the rehabilitation device of the present invention, the head mounted unit is disposed on the surface of the scalp of the subject prior to the subject's performing rehabilitation. In addition, the motion sensor is attached to a part (for example, the head) of the subject's body that is different from the paralyzed part (for example, the hand). Furthermore, the subject causes the exercises (exercises of the hand) of the paralyzed part carried out as rehabilitation to occur. At this time, the brain measurement data acquisition unit acquires brain measurement data concerning brain activity from the head mounted unit, and the body measurement data acquisition unit also acquires body measurement data concerning bodily movements of the part (head) of the subject's body from the motion sensor. Thus, when there is detection of bodily movement (movement of the head rather than the hand) that does not correspond to the exercises targeted by the rehabilitation, the warning unit gives a warning to stop the bodily movement of the part of the subject's body (head). As a result, the subject can be made to understand that there is bodily movement of the part (head) of the body and stop the bodily movement of the part (head) of the body.

On the other hand, when there is no bodily movement (movement of the head rather than the hand) that does not correspond to the exercises targeted by the rehabilitation, the warning unit does not give a warning. Therefore, the subject continues with the exercise (movement of the hand) that is targeted by the rehabilitation.

As per the above, accurate brain measurement data into which artifacts and noise do not enter can be obtained according to the rehabilitation device of the present invention, and effective rehabilitation can be carried out by the subject.

### Means for Solving Other Problems and Effects

In addition, the exercises for the rehabilitation device of the present invention are ones that move the hand, which is the paralyzed part of the subject, and the motion sensor may be attached to the head of the subject.

Furthermore the head mounted unit of the rehabilitation device the present invention is a light transmitting and receiving unit that has at least one light transmitting probe disposed on the surface of the scalp and at least one light receiving probe disposed on the surface of the scalp. The brain measurement data acquisition unit may acquire brain measurement data by carrying out control so as to have the light transmitting probe irradiate the surface of the scalp with light and also have the light receiving probe detect light released from the surface of the scalp.

### BRIEF DESCRIPTION OF THE DRAWINGS

FIG. 1 is a block diagram showing a constitution of a rehabilitation device, which is an embodiment of the present invention.

FIG. 2 is a plan view showing an example of a holder into which light transmitting probes and light receiving probes are inserted.

FIG. 3 is a flowchart for describing an example of a rehabilitation method.

FIG. 4 is a drawing showing the relationship between a measurement site and a pair of a light transmitting probe and a light receiving probe.

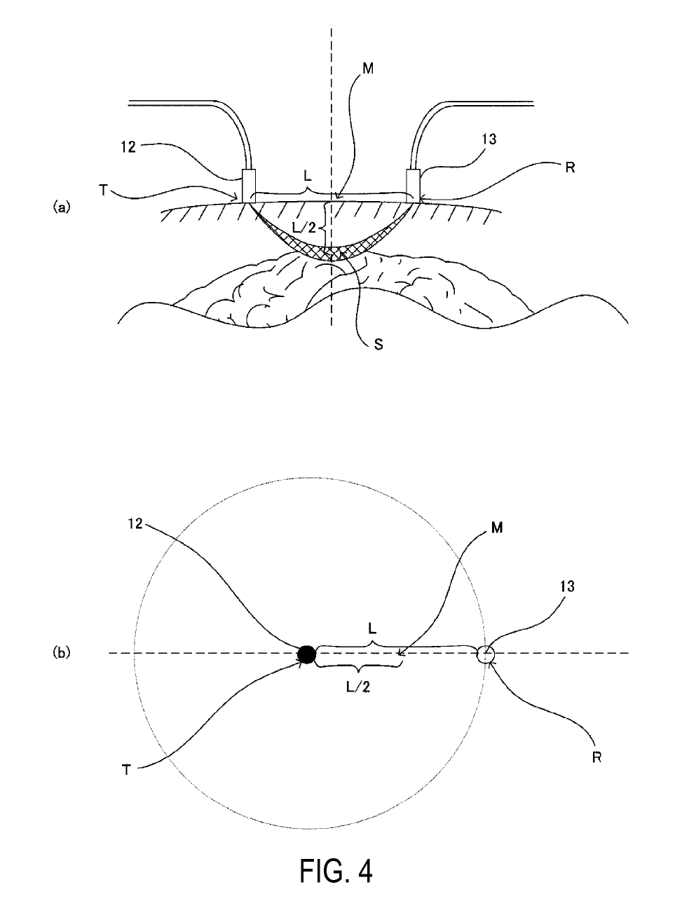

FIG. 5 is a block diagram showing an example of a schematic constitution of a conventional near-infrared spectrometer.

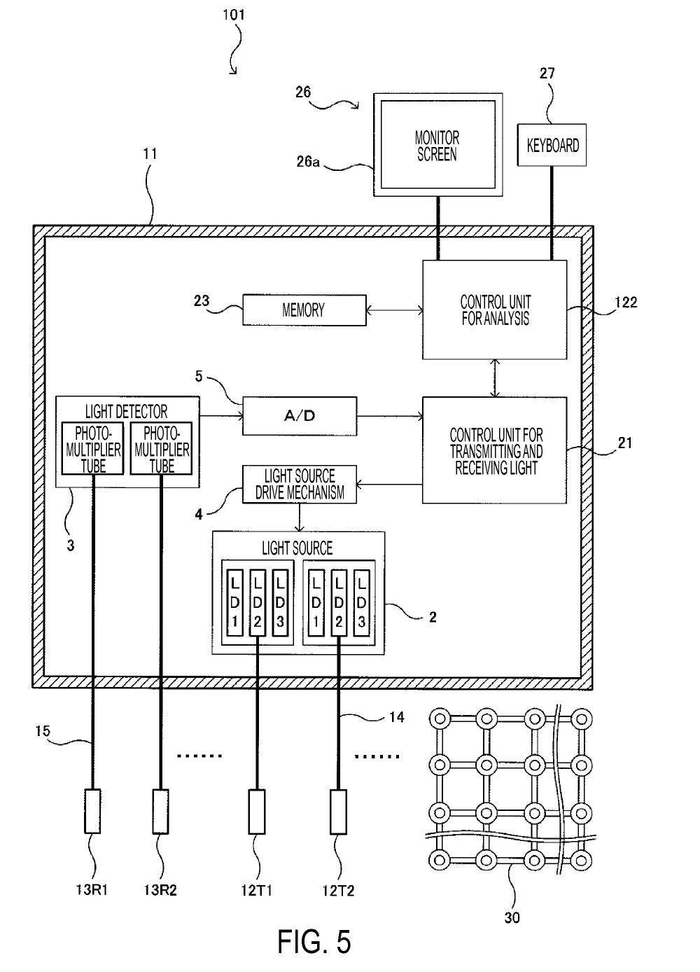

### MODE FOR CARRYING OUT THE INVENTION

In the following, embodiments of the present invention are described using the drawings. Moreover, the present invention is not limited to the embodiments described below, and it need not be mentioned that various embodiments are included within a range that does not depart from the essence of the present invention.

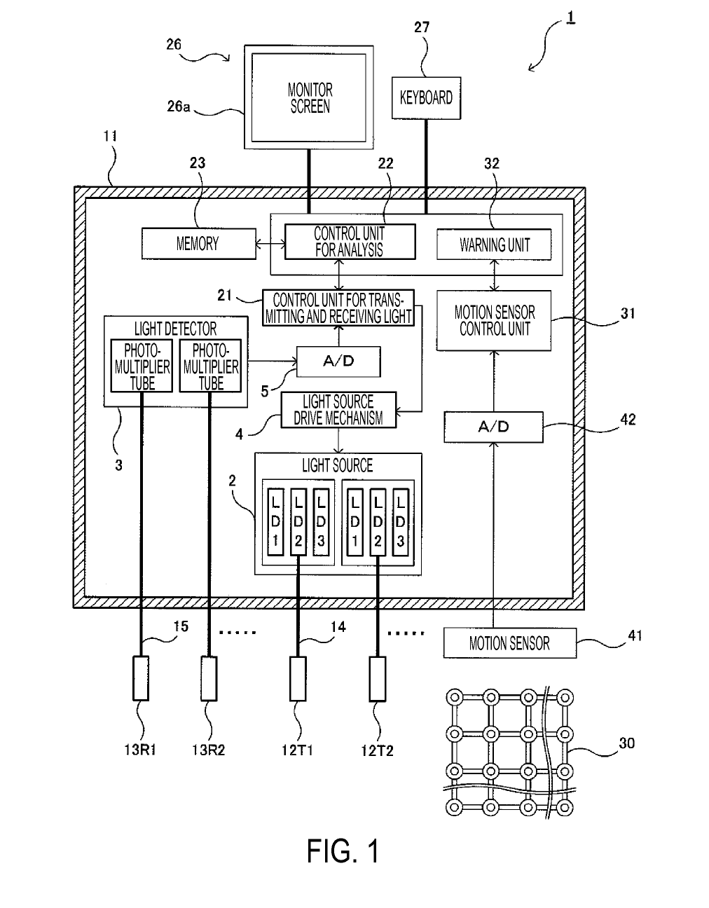

FIG. 1 is a block diagram showing a constitution of a rehabilitation device, which is an embodiment of the present invention. Moreover, the same element numbers are used for the same elements in the near-infrared spectrometer 101.

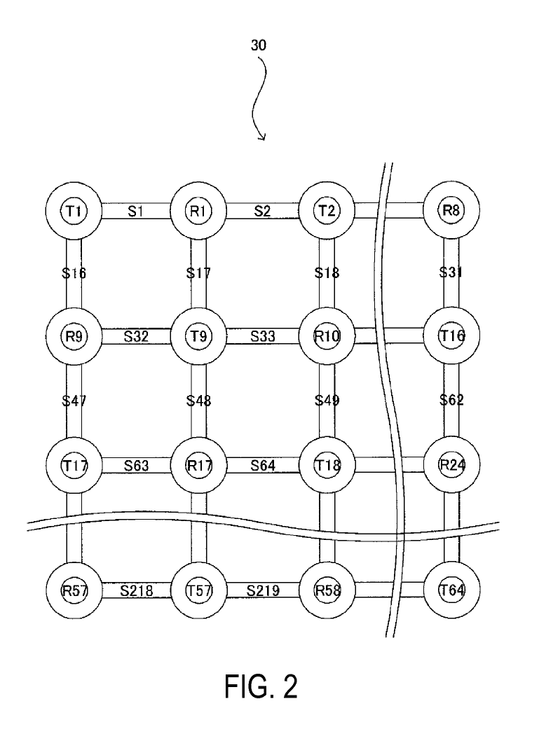

A rehabilitation device 1 has a cuboid shaped housing 11. Inside the housing 11, there are provided: a light source 2 that irradiates light, a light source drive mechanism 4 that drives the light source 2, a light detector 3 that detects light, A/Ds (A/D converters) 5, 42, a control unit 21 for transmitting and receiving light (brain measurement data acquisition unit), a control unit 22 for analysis (determination unit), a motion sensor control unit 31 (body measurement data acquisition unit), alarm unit 32 and memory 23. In addition, outside the housing 11, there are provided: 64 light transmitting probes 12, 64 light receiving probes 13, 64 light transmitting optical fibers 14, 64 light receiving optical fibers 15, a motion sensor 41 a display device 26 that has a monitor screen 26a and the like and a keyboard (input device) 27. In addition, the rehabilitation device 1 is provided with a holder 30, as shown by example in FIG. 2.

Here, in order to determine whether or not the brain of a patient (subject) can recall brain activity corresponding to exercises targeted by the rehabilitation in the present embodiment, values for the product [oxyHb] of the oxyhemoglobin concentration and the optical path length, the product [deoxyHb] of the deoxyhemoglobin concentration and the optical path length and the product ([oxyHb]+[deoxyHb]) of the total hemoglobin concentration and the optical path length, which are measured by near infrared light of three different wavelengths and for brain measurement data, are used. In addition, the patient recalls an operation alternately closing and opening a hand (part of the body) that has been paralyzed by an injury such as a stroke, or the like, as the exercises targeted by rehabilitation. Also, by recalling, the patient carries out the operation of alternately closing and opening the hand that has been paralyzed.

Thus, brain measurement data showing brain activity corresponding to the exercises targeted by the rehabilitation is stored in advance in the memory 23 as data for determination for determining whether or not brain activity corresponding to the exercises targeted by the rehabilitation is recalled. The data for determination is, for example, the values for the product [oxyHb] of the oxyhemoglobin concentration and the optical path length, the product [deoxyHb] of the deoxyhemoglobin concentration and the optical path length and the product ([oxyHb]+[deoxyHb]) of the total hemoglobin concentration and the optical path length at a brain coordinate location (x, y, z), which have been characterized by measuring the values for the product [oxyHb] of the oxyhemoglobin concentration and the optical path length, the product [deoxyHb] of the deoxyhemoglobin concentration and the optical path length and the product ([oxyHb]+[deoxyHb]) of the total hemoglobin concentration and the optical path length at various brain coordinate locations when the exercises targeted by the rehabilitation are carried out with a healthy subject that have been stored.

For example, when a brain coordinate location (x₁, y₁, z₁) is determined to be characteristic when a healthy subject carries out the action of alternately closing and opening the hand, the values for the product [oxyHb] of the oxyhemoglobin concentration and the optical path length, the product [deoxyHb] of the deoxyhemoglobin concentration and the optical path length and the product ([oxyHb]+[deoxyHb]) of the total hemoglobin concentration and the optical path length at the brain coordinate location (x₁, y₁, z₁) are stored as brain measurement data obtained when the action of alternately closing and opening the hand is carried out. Moreover, the brain coordinate location (x, y, z) is a value shown by Talairach coordinates or MNI coordinates.

When the patient engages in rehabilitation, the control unit 22 for analysis locates the product [oxyHb] of the oxyhemoglobin concentration and the optical path length, the product [oxyHb] of the oxyhemoglobin concentration and optical path length and the product ([oxyHb]+[deoxyHb]) of the total hemoglobin concentration and the optical path length from the intensity of the light of the various wavelengths (oxyhemoglobin absorption wavelength and deoxyhemoglobin absorption wavelength) passing through using relational equations (1), (2) and (3) based on the total of 232 sets of information on the amount of light received A(λ₁), A(λ₂) and A(λ₃). Furthermore, the control unit 22 for analysis determines whether or not the brain of the patient can recall the brain activity corresponding to the action of alternately closing and opening the hand (exercise targeted by the rehabilitation) based on the product [oxyHb] of oxyhemoglobin concentration and the optical path length (brain measurement data). For example, a determination is made as to how close the brain measurement data value is to the data for determination by comparing the values for the product [oxyHb] of the oxyhemoglobin concentration and the optical path length, the product [deoxyHb] of the deoxyhemoglobin concentration and optical path length and the product ([oxyHb]+[deoxyHb]) of the total hemoglobin concentration and the optical path length at the brain coordinate location (x, y, z) and the data for determination. If the values for the brain measurement data are within a prescribed range of the data for determination at this time, the brain of the patient is said to be able to recall the brain activity corresponding to the action of alternately closing and opening the hand (exercise targeted by the rehabilitation). Furthermore, the control unit 22 for analysis displays an image showing the determination result on the display screen 26a. Thus, the patient can engage in rehabilitation effectively while observing the determination results.

Furthermore, in the present embodiment, a motion sensor 41 is provided for attaching to a part (for example, the head, arm or the like) of the patient's body that is different from the paralyzed part (for example, the hand) such that determination results are that are obtained by means of accurate brain measurement data into which artifacts and noise did not enter are displayed. The motion sensor 41 outputs body measurement data B to the motion sensor control unit 31 via the A/D 42 by detecting the body measurement data B, which shows movement and, for example, is a six axis motion sensor, with a three axis acceleration sensor and three axis gyro sensor, or the like.

The motion sensor control unit 31 carries out control for obtaining the body measurement data B detected by the motion sensor 41 in a prescribed time interval (for example, an interval of one second) when the patient is engaging in rehabilitation.

The warning unit 32 provides a warning such that bodily movement by the patient of part of the body is stopped when bodily movement that does not correspond to the exercises targeted by the rehabilitation is detected based on the body measurement data B when the patient is engaged in rehabilitation. For example, when the motion sensor 41 is attached to the head of the patient and, regardless of the patient's recall of the action of alternately closing and opening the hand, the body measurement data B exceeds a threshold value, an image of “Do not move your head” is displayed on the monitor screen 26a. On the other hand, when the body measurement data B is equal to or less than the threshold value, the image showing the warning is not displayed on the monitor screen 26a.

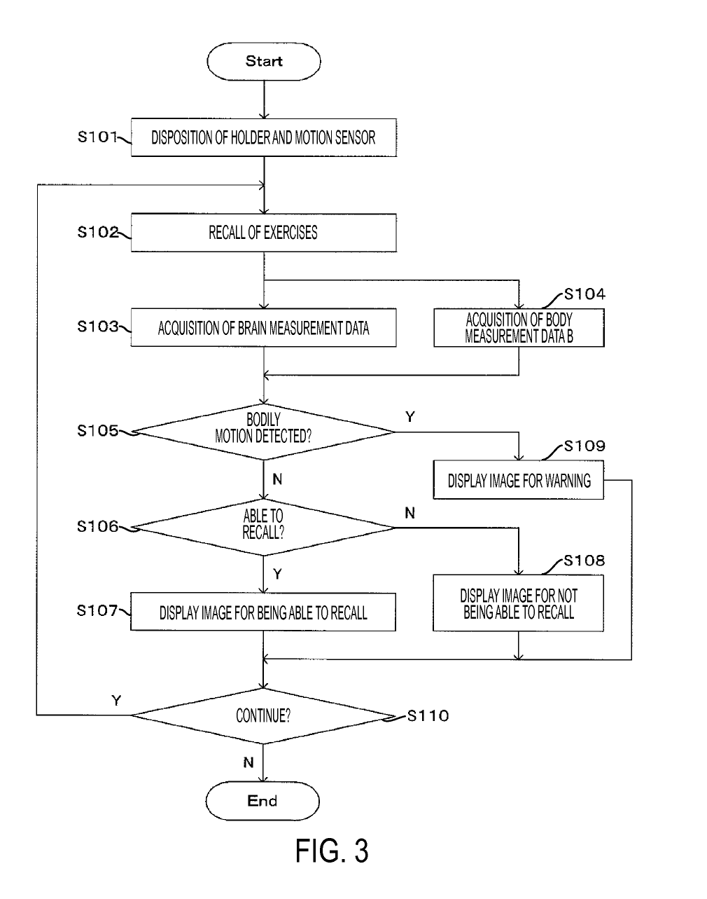

Here, a rehabilitation method for a patient engaging in rehabilitation by means of the rehabilitation device 1 will be described. FIG. 3 is a flowchart for describing one example of the rehabilitation method.

First, in the processing for Step S101, a physician disposes the holder 30 on the surface of the scalp of the patient and also attaches the motion sensor 41 to the head (part of the body different from the paralyzed part (hand)).

Next, in the processing for Step S102, an instruction to recall the movement for alternately closing and opening the hand (exercises targeted by the rehabilitation) is given to the patient.

Next, in the processing for Step S103, the control unit 21 for transmitting and receiving light outputs the drive signal for transmitting light to one light transmitting probe 12 to the light source drive mechanism 4 at a prescribed time based on a control table stored in the memory 23, and a light receiving signal (brain measurement data) for light received by a light receiving probe 13 is detected by the light detector 3.

Next, at the same time as the processing for Step 5103 is executed, the motion sensor control unit 31 obtains the body measurement data B detected by the motion sensor 41 in the processing for Step 104.

Next, in the processing for Step S105, the warning unit 32 determines whether or not there is motion of the head (bodily movement that does not correspond to the exercises targeted by the rehabilitation). When it is determined that there is no motion of the head (bodily movement that does not correspond to the exercises targeted by the rehabilitation), in the processing for Step S106, the control unit 22 for analysis determines whether or not the brain of the patient can recall the brain activity corresponding to the action of alternately closing and opening the hand (exercises targeted by the rehabilitation) based on the product [oxyHb] of oxyhemoglobin concentration and the optical path length and the like.

When it is determined that the brain of the patient can recall the brain activity corresponding to the operation of alternately closing and opening the hand (exercises targeted by the rehabilitation), in the processing for Step S107, the control unit 22 for analysis does not display an image giving a warning on the monitor screen 26a and displays an image showing “You are recalling very well.”

On the other hand, when it is determined that the brain of the patient cannot recall the brain activity corresponding to the action of alternately closing and opening the hand (exercises targeted by the rehabilitation), in the processing for Step S108, the control unit 22 for analysis displays an image showing “Recall not accomplished” on the monitor screen 26a.

On the other hand, in the processing for Step S105, when it is determined that there is movement of the head (bodily movement that does not correspond to the exercises targeted by the rehabilitation), the warning unit 32 displays an image showing a warning on the monitor screen 26a such that the movement of the head (bodily movement that does not correspond to the exercises targeted by the rehabilitation) is stopped, in the processing for Step S109.

Furthermore, after the execution of any of the processing for the processing for Step S107, the processing for Step S108 or the processing for Step S109, a judgment is made as to whether or not the patient continues to engage in the rehabilitation in the processing for Step S110. When it is determined that the patient continues engaging in the rehabilitation, there is a return to the processing in Step S102. In other words, until there is a determination that the patient stops engaging in rehabilitation, the processing for Step S102-Step S110 is repeated.

On the other hand, when there is a determination for stopping the patient from engaging in the rehabilitation, this flowchart ends.

As per the above, according to the rehabilitation device 1, accurate brain measurement data into which artifacts and noise do not enter can be obtained, and the patient can engage in rehabilitation effectively.

### Other Embodiments

(1) In the rehabilitation device 1 described above, the holder 30 having 64 light transmitting probes 12 and 64 light receiving probes 13 was shown as a nonlimiting example, but there may be a holder with a different number, for example nine light transmitting probes and nine light receiving probes.

(2) In the rehabilitation device 1 described above, a constitution using values for the product [oxyHb] of the oxyhemoglobin concentration and the optical path length, the product [deoxyHb] of the deoxyhemoglobin concentration and optical path length and the product ([oxyHb]+[deoxyHb]) of the total hemoglobin concentration and the optical path length measured by near infrared light of three different wavelengths and as the brain measurement data was shown, but there may be a constitution using brain measurement data obtained from an electroencephalogram (EEG) or the like.

(3) In the rehabilitation device 1 described above, the constitution in which one motion sensor 41 was attached to the head of the patient was shown, but the same may be attached to an arm of the patient, and furthermore, there may be a constitution in which a plurality of motion sensors 41 are attached to the arms and legs of the patient.

(4) In the rehabilitation device 1 described above, a constitution in which the brain of the patient was said to be able to recall brain activity corresponding to exercises targeted by the rehabilitation when the brain measurement data value was within a prescribed range of the data for determination, but there may be a constitution in which the extent to which the brain of the patient could recall brain activity is shown by a graph.

### INDUSTRIAL APPLICABILITY

The present invention can be used in rehabilitation devices for rehabilitation of patients (subjects) in which part of the body has been paralyzed due to a stroke or the like.

### EXPLANATION OF THE ELEMENTS

1: rehabilitation device
12: light transmitting probe
13: light receiving probe
21: control unit for transmitting and receiving light (brain measurement data acquisition unit)
22: control unit for analysis (determination unit)
30: holder (head mounted unit)
31: motion sensor control unit (body measurement data acquisition unit)
32: warning unit
41: motion sensor
T: light transmission point
R: light receiving point
M: measurement point

1. A rehabilitation device comprising:
a head mounted unit disposed on the surface of the scalp of a subject;
a brain measurement data acquisition unit that obtains brain measurement data concerning brain activity by controlling the head mounted unit;
a determination unit that determines whether or not the brain of the subject can recall brain activity corresponding to exercises of a paralyzed part targeted by rehabilitation based on the brain measurement data;
a motion sensor attached to part of the subject's body other than the paralyzed part;
a body measurement data acquisition unit that obtains body measurement data concerning bodily movement of the part of the subject's body from the motion sensor; and
a warning unit that gives a warning to stop bodily movement of the part of the subject's body when, based on the body measurement data, there is detection of bodily movement that does not correspond to the exercises targeted by the rehabilitation.

2. The rehabilitation device according to claim 1, wherein
the exercises are ones for moving a hand, which is a paralyzed part of the subject, and
the motion sensor is attached to the head of the subject.

3. The rehabilitation device according to claim 1, wherein:
the head mounted unit is a light transmitting and receiving unit that has at least one light transmitting probe disposed on the surface of the scalp and at least one light receiving probe disposed on the surface of the scalp, and
the brain measurement data acquisition unit acquires brain measurement data by carrying out control so as to have the light transmitting probe irradiate the surface of the scalp with light and also have the light receiving probe detect light released from the surface of the scalp.

4. The rehabilitation device according to claim 2, wherein:
the head mounted unit is a light transmitting and receiving unit that has at least one light transmitting probe disposed on the surface of the scalp and at least one light receiving probe disposed on the surface of the scalp, and
the brain measurement data acquisition unit acquires brain measurement data by carrying out control so as to have the light transmitting probe irradiate the surface of the scalp with light and also have the light receiving probe detect light released from the surface of the scalp.
